(19) United States
(12) Patent Application Publication
(10) Pub. No.: US 2014/0316177 A1
(43) Pub. Date: Oct. 23, 2014

(54) CATALYST FOR USE IN PRODUCTION OF HYDROCARBONS

(71) Applicants: Qingjie Ge, Dalian (CN); Xiangang Ma, Dalian (CN); Chuanyan Fang, Dalian (CN); Hengyong Xu, Dalian (CN)

(72) Inventors: Qingjie Ge, Dalian (CN); Xiangang Ma, Dalian (CN); Chuanyan Fang, Dalian (CN); Hengyong Xu, Dalian (CN)

(73) Assignees: DALIAN INSTITUTE OF CHEMICAL PHYSICS, CHINESE ACADEMY OF SCIENCES, DALIAN LIAONING PROVINCE (CN); BP P.L.C., LONDON (GB)

(21) Appl. No.: 14/113,052

(22) PCT Filed: Apr. 21, 2011
(86) PCT No.: PCT/CN11/00696

§ 371 Date: Feb. 6, 2014

(51) Int. Cl. B01J 29/14; C07C 5/03; B01J 29/12

(52) U.S. Cl. CPC B01J 29/146; B01J 29/126; C07C 5/03
USPC 585/254; 502/60; 502/79; 502/74; 502/64

(57) ABSTRACT

A modified catalyst is described which can be used as a dehydration/hydrogenation catalyst in a multistage catalyst system for the catalysed production of saturated hydrocarbons from carbon oxides and hydrogen. The modified catalyst comprises: an acidic substrate comprising an M1-zeolite or M1-silicoalumino phosphate (SAPO) catalyst, where M1 is a metal; and a modifier including a metal M2. M2 comprises an alkali metal or alkaline earth metal. In examples described the modifier includes a Group II metal, for example Ca.

This invention relates to catalysts. In particular, but not exclusively, aspects of this invention relate to catalysts for use in a process for the production of hydrocarbons. Examples of the invention relate to catalysts for use in the production of liquefied petroleum gas from synthesis gas. Aspects of the invention also find application in relation to the production of liquid fuels for example gasoline.

In recent years, the dominance of natural gas and petroleum as feedstocks has diminished. New feedstocks such as tar sands, coal, biomass and municipal waste have been increasing in importance. The diversity of feedstocks has driven the development of synthesis gas (syngas) routes to replace conventional routes to hydrocarbons, in particular liquid hydrocarbons, from natural gas and petroleum.

Liquefied petroleum gas (LPG), a general description of propane and butane, has environmentally relatively benign characteristics and widely been used as a so-called clean fuel. Conventionally, LPG has been produced as a byproduct of liquefaction of natural gas, or as a byproduct of refinery operations. LPG obtained by such methods generally consists of mainly propane and n-butane mixtures. Alternative sources for LPG would be desirable. Synthesis of LPG from syngas is potentially a useful route as it would allow for the conversion of diverse feedstocks, for example natural gas, biomass, coal, tar sands and refinery residues.

One synthesis route to hydrocarbons uses the Fischer-Tropsch synthesis reaction. However, this can be disadvantageous in that the product hydrocarbons will follow Anderson-Schulz-Flory distribution, and as a result the selectivity to LPG would be relatively limited. In particular, such a process would generally produce significant amounts of undesirable methane together with higher linear hydrocarbons.

Therefore a new synthesis method to produce LPG which overcame or at least mitigated one or more of these or other disadvantages would be desirable.

Processes exist for selectively converting syngas to for example methane or methanol. The conversion of methanol to C₂ and C₃ products as exemplified in the methanol to olefins (MTO) and methanol to propylene (MTP) processes is well known, for example as described in U.S. Pat. No. 6,613,951. However, in some cases, the selectivity may be limited and products may consist predominantly of C₂ and C₃ olefins.

The methanol to gasoline (MTG) process as developed by Mobil allows access to a mixed product rich in aromatics and olefins.

Neither of these processes is selective to LPG or higher saturated hydrocarbons.

Recently, several investigations have been made relating to a process for the production LPG from syngas. Some investigations involve multifunctional catalyst systems. For example Zhang Q, et al. Catalysis Letters Vol 102, Nos 1-2 Jul. 2005, describes hybrid catalysts based on Pd—Ca/SiO₂ and zeolite, and on Cu—Zn/zeolite. Both hybrid catalyst systems were reported to have reasonable selectivity to LPG but the Cu—Zn/zeolite was reported to be deactivated rapidly under the high temperature reaction conditions required, and while the Pd—Ca/SiO₂ system was found to be more stable, it had a relatively low activity.

Qingjie Ge et al, Journal of Molecular Catalysis A: Chemical 278 (2007) 215-219, describes the reaction of synthesis gas to produce LPG using a mixed catalyst system in a single bed comprising a Pd—Zn—Cr methanol synthesis catalyst and a Pd-loaded zeolite for dehydration of methanol and dimethyl ether (DME). Reaction temperatures used were more than 330 degrees C. and the high reaction temperatures were reported to improve selectivity to LPG. However, despite advantageous synergy reported between the two catalysts, the lifetime of the catalyst was found to be an issue. Coking of the catalyst was thought to decrease the performance of the catalyst with time on stream. Also, the described catalyst has a Pd content of 0.5 wt %, and it would be desirable to reduce the amount of precious metal required.

The selective synthesis of LPG from syngas may be carried out over a hybrid catalyst comprising a methanol synthesis catalyst and modified zeolite. The methanol synthesis catalyst used may for example be a Cu-based methanol synthesis catalyst, and zeolites may be for example Y or β zeolite. For example, Li et al reported (JP2009195815A) a hybrid catalyst composed of Cu—ZnO methanol synthesis catalysts with Pd-modified β zeolite for syngas to LPG conversion in a slurry-bed reactor. CN 101415492A describes Cu—ZnO/Pd-β catalysts for syngas to LPG conversion.

In such systems and/or other systems, however, it is believed that the sintering of Cu in methanol synthesis catalyst and coke deposition on zeolite are the two major factors for the deactivation of the hybrid catalyst. At low temperature, the low cracking rate of heavy hydrocarbons may result in more coke formation. Thus the zeolite may deactivate relatively quickly. Coke deposition may also decrease significantly the LPG selectivity in hydrocarbons. This may also have some effect on the CO conversion. On the other hand, the use of high temperature can lead to fast sintering of Cu which significantly reduces CO conversion. Cu-based methanol synthesis catalyst is a cheap commercial product, so the stability of zeolite should be improved by restraining coke formation at low temperature in order to enable the process of LPG synthesis from syngas.

A catalyst which had improved stability and/or lifetime compared with conventional catalysts would be desirable.

According to an aspect of the invention there is provided a modified catalyst for use as a dehydration/hydrogenation catalyst in a multi-stage catalyst system for the catalysed production of saturated hydrocarbons from carbon oxides and hydrogen, the modified catalyst comprising:

an acidic substrate comprising an M1-zeolite or M1-silicoalumino phosphate (SAPO) catalyst, where M1 is a metal; and

a modifier including a metal M2,

wherein M2 comprises an alkali metal or alkaline earth metal.

Without wishing to be bound by any particular theory, it is thought that coke deposition takes place relatively easily on strong acid sites of the acidic substrate. The inventors have identified that the stability of a hybrid catalyst might be improved if the strong acid sites of the acidic substrate were to be weakened. According to aspects of the present invention, such weakening could be effected by modification of the acidic substrate for example by the addition of the modifier.

Where reference is made herein to an acidic substrate, preferably the substrate is a Bronsted acid.

The modified catalyst may have been prepared for example by a method described herein. However, some aspects of the invention extend to the case in which a “modified catalyst” is obtained by other methods or from other sources. Such catalyst will preferably comprise an acidic substrate comprising an M1-zeolite or M1-silicoalumino phosphate (SAPO) catalyst, where M1 is a metal; and a modifier including a metal M2, M2 comprising an alkali metal or alkaline earth metal. Thus aspects of the invention extend to such catalyst compositions irrespective of their source or method of preparation.

Suzuki, Applied Catalysis 39 (1988) 315-324 describes preparing a catalyst for converting methanol to hydrocarbons, for example alkenes. The catalyst preparation is from aqueous solution and includes adding Ca₂P₂O₇ to ZSM-5 at a high wt % of up to 50%. The authors report that the catalyst obtained shows an improved coke resistance and catalyst life.

Zhang, Ind Eng Chem Res (2010) 49 2103-2106 describes Ca loading into HZSM-5 zeolites and the use of the catalysts prepared for MTO reactions to form olefins. The loss of the Bronsted acid sites on the zeolites is discussed.

U.S. Pat. No. 4,289,710 of Union Carbide Corporation describes a Pd methanol synthesis catalyst using carriers containing calcium. U.S. Pat. No. 4,547,482 of Mitsubishi Gas Chemical Company Inc describes the use of Ca in the formulation of a Cu/ZnO methanol synthesis catalyst.

U.S. Pat. No. 7,297,825 describes a hybrid catalyst for syngas to LPG including a Pd-based methanol synthesis catalyst and a beta-zeolite. In examples described, Ca is added to the methanol synthesis catalyst.

The inventors have identified that an M1-acidic substrate catalyst comprising a modifier comprising M2, for use as a dehydration/hydrogenation catalyst can give improved resistance to coking of the catalyst in a dehydration/hydrogenation process. Furthermore, as discussed further below, the inventors have identified that the modification of an M1-zeolite catalyst by the addition of M2 can improve resistance to coking of the catalyst. Also, and as discussed further below, the inventors have additionally identified that a hybrid catalyst including the modified catalyst, for example including the modified catalyst and a carbon oxide(s) catalyst can have improved resistance to coking of the catalyst. Examples below describe how the modified catalyst may be for example mixed with a methanol synthesis catalyst to form a hybrid catalyst.

Preferably the SAPO comprises a crystalline microporous silicoalumino phosphate composition. Silicoalumino phosphates are known to form crystalline structures having micropores which compositions can be used as molecular sieves for example as adsorbents or catalysts in chemical reactions. SAPO materials include microporous materials having micropores formed by ring structures, including 8, 10 or 12-membered ring structures. Some SAPO compositions which have the form of molecular sieves have a three-dimensional microporous crystal framework structure of PO₂⁺, AlO₂⁻, and SiO₂ tetrahedral units. The ring structures give rise to an average pore size of from about 0.3 nm to about 1.5 nm or more. Examples of SAPO molecular sieves and methods for their preparation are described in U.S. Pat. No. 4,440,871 and U.S. Pat. No. 6,685,905 (the content of which are incorporated herein by reference).

Preferably the modifier comprises a group II metal. Preferably the modifier comprises a source of a group II metal ion. The modifier may include Ca. Thus M2 may comprise Ca. The modifier may include a single component or a mixture of two or more components. The modifier may include a source of one or more group I or group II metal ions.

In, a method of preparation of the modified catalyst, M2 is preferably added in the form of a soluble salt. Preferred salts include acetates, formates, propionates, nitrates, oxalates and adipates.

The acidic substrate may comprise one or more from the group comprising Y zeolite, β zeolite, ZSM-5 and SAPO-5, SAPO-34 and mordenite. The acidic substrate may comprise two or more such components from the group.

M1 preferably comprises a hydrogenation catalyst. The hydrogenation catalyst preferably comprises a metal chosen from the group comprising Pd, Pt, Rh, Ru, and Cu.

The weight percent of metal M1 added to the acidic substrate in the method of preparation of the catalyst is from about 0.1 wt % to about 2 wt %, for example from about 0.5 wt % to about 1 wt %.

The weight percent of modifier, for example calcium, added to the acidic substrate is preferably chosen such that the concentration of strong acidic sites of the support is reduced. Preferably the concentration of weak acid sites of the support is not significantly reduced.

For example, the acidity of the substrate can be measured using NH₃-TPD analysis as described in Zhang, Ind Eng Chem Res (2010) 49 2103-2106. Preferably at least 25% of the strong acid sites, for example at least 50% of the strong acid sites, for example at least strong 75% of the sites are neutralized by the addition of the modifier. Preferably less than 100% of the acid sites are neutralized. Strong and week acidity could be measured using NH₃-TPD analysis. The NH₃ desorption peak of weak acidity was at relatively low temperature, and the NH₃ desorption peak of strong acidity was at relatively high temperature. In some examples, the border between strong acidity and weak acidity was about 300 degrees C. The amount of strong and weak acidity could be calculated from a measurement of peak area.

Preferably the weight percent of alkali or alkali earth metal added to the acidic substrate relative to the acidic substrate is about from 0.1 wt % to about 2 wt %, for example from about 0.5 wt % to about 1 wt % where M2 is Ca and the substrate is Y-zeolite. It will be understood that comparable wt % may be preferred for other metals M2 and/or other substrates.

In preferred examples, for example where M1 is Pd and M2 is Ca, the ratio of metal M2 to metal M1 by weight is between from about 0.1 to about 10, for example between from about 1 to 2.

As discussed above, in some applications of aspects of the invention, the modified catalyst is used in combination with an additional catalyst, for example a carbon oxide(s) conversion catalyst. Thus aspects of the invention provide a catalyst system including the modified catalyst and a carbon oxide(s) conversion catalyst. The catalyst system may comprise a two-stage catalyst system, for example in which the two stages of the system are separate. The two-stage catalyst system may be a part of a multi-stage catalyst system.

Thus a further aspect of the invention provides a multi-stage catalyst system for use as a dehydration/hydrogenation catalyst in the catalysed production of saturated hydrocarbons from carbon oxides and hydrogen, the catalyst system comprising a first stage comprising a carbon oxide(s) conversion catalyst, and a second stage comprising a modified catalyst comprising:

an M1-zeolite or M1-SAPO catalyst, where M1 is a metal; and

a modifier comprising a metal M2,

wherein M2 is an alkali metal or alkaline earth metal.

The multi-stage catalyst system is preferably used as physically separate stages, or physically segmented stages, although other options are possible.

The carbon oxides conversion methanol synthesis catalyst may be active to produce dimethyl ether (DME), for example to produce DME in the first stage where a two-stage or multi-stage system is used, or for a hybrid catalyst, to produce DME in the catalysed conversion process. In some examples, both methanol and DME may be produced in the process.

The production of methanol from carbon oxide(s) and hydrogen is equilibrium limited. The production of DME direct from carbon oxide(s) and hydrogen is less equilibrium limited. Pressure can be used to increase the yield, as the reaction which produces methanol exhibits a decrease in volume, as disclosed in U.S. Pat. No. 3,326,956. Improved catalysts have allowed viable rates of methanol formation to be achieved at relatively low reaction temperatures, and hence allow commercial operation at lower reaction pressures. For example a CuO/ZnO/Al₂O₃ conversion catalyst may be operated at a nominal pressure of 5-10 MPa and at temperatures ranging from approximately 150 degrees C. to 300 degrees C. However, at higher reaction temperatures, reduction in catalyst lifetime has commercially been found to be a problem. A low-pressure, copper-based methanol synthesis catalyst is commercially available from suppliers such as BASF and Haldor-Topsoe. Methanol yields from copper-based catalysts are generally over 99.5% of the converted carbon oxide(s) present. Water is a by-product of the conversion of CO₂ to methanol and the conversion of synthesis gas to C₂ and C₂₊ oxygenates. In the presence of an active water gas-shift catalyst, such as a methanol catalyst or a cobalt molybdenum catalyst, the water equilibrates with the carbon monoxide to give CO₂ and hydrogen.

Recently, to seek to overcome the equilibrium limitation of the methanol synthesis catalyst, direct syngas-to-DME processes have been developed. These processes are thought to proceed via a methanol intermediate which is etherified by an added acid functionality in the catalyst, for example as described in PS Sai Prasad, et al., Fuel Processing Technology Volume 89, Issue 12, December 2008, p 1281-1286.

The carbon oxide(s) conversion catalyst may be provided together with the modified catalyst in a hybrid catalyst. Thus a methanol synthesis catalyst and the modified catalyst will be present together in a hybrid catalyst. The hybrid catalyst may for example include a mechanical mixture of the modified catalyst and a methanol synthesis catalyst.

Therefore, a further aspect of the invention provides a hybrid catalyst for the catalysed production of saturated hydrocarbons from carbon oxides and hydrogen, hybrid catalyst including:

a carbon oxide(s) conversion catalyst, and

a modified catalyst comprising:

a dehydration/hydrogenation catalyst including an M1-zeolite or M1-SAPO catalyst,

where M1 is a metal; and

a modifier including a metal M2,

wherein M2 is an alkali metal or alkaline earth metal.

The carbon oxide(s) conversion catalyst may for example comprise a methanol synthesis catalyst. The methanol synthesis catalyst may be any appropriate composition. In preferred examples, the catalyst includes Cu—ZnO-[Sup], Pd-[Sup] and Zn—Cr-[Sup], where [Sup] is preferably a support composition for example including Al₂O₃, SiO₂, and/or zeolite.

The hybrid catalyst may be prepared by any appropriate method, for example by a mechanical mixing method with methanol synthesis catalyst and modified zeolite. The weight percent of modified catalyst in the hybrid catalyst may be for example from about 20% to 80%, for example from about 40% to 70%.

Also provided by the invention is a modified catalyst for use in the catalysed production of saturated hydrocarbons from carbon oxides and hydrogen, the modified catalyst comprising:

an M1-SAPO catalyst, where M1 is a metal; and

a modifier including a metal M2,

wherein M2 is an alkali metal or alkaline earth metal.

According to the invention, there is also provided a method or methods of preparing any of the catalysts described herein.

The method may include the step of adding the metal M1 and the modifier substantially simultaneously to the acidic substrate. In other examples, the metal M1 is preferably added before the metal M2.

It has been identified by the inventors that by adding the metal M1 before or simultaneously with the metal M2, the modified catalyst can have in some examples improved resistance to coking, while retaining an acceptable catalytic activity. It has been identified that if the modifier M2 is added before the metal M1, in some cases the metal M2 can limit the amount of metal M1 which can be loaded into the catalyst, thus reducing the activity of the modified catalyst. For example, in a method of forming the modified catalyst in which 0.5 wt % Ca was impregnated onto a Y zeolite by incipient-wetness impregnation. Subsequently, Pd was added by an ion-exchange method. It was seen that so little Pd was exchanged onto the Y zeolite after Ca impregnation that the hydrogenation ability of the catalyst decreased significantly compared with other catalysts. The poor hydrogenation ability could not restrain olefins polymerizing to form coke, and that resulted in the quick deactivation of the hybrid catalyst Cu—Zn—Al/0.5IMPCa-IEPd—Y.

This feature is of particular importance and is provided independently. Thus a further aspect of the invention provides a method of preparing a modified catalyst for use in the catalysed production of saturated hydrocarbons, the catalyst comprising a metal M1 and an acidic substrate selected from a zeolite and/or a silicoalumino phosphate (SAPO), and the modifier including a metal M2, wherein M2 is an alkali metal or alkaline earth metal, the method including the step of adding the metal M1 and the modifier to the acidic substrate, wherein the metal M1 is added to the acidic substrate before or at substantially the same time as the modifier.

The modifier and metal may be applied using the same or different methods. The metal M1 and/or the modifier may be added to the acidic substrate by an ion exchange method. The temperature for the ion-exchange method where used may be for example from about 30 to 80 degrees C., for example from about 50 to 60 degrees C. Alternatively, or in addition, the metal M1 and/or the modifier are added to the acidic substrate by an incipient wetness impregnation method. The incipient wetness impregnation method is a known method for impregnating catalyst supports. It comprises for example the steps of adding a solution of catalyst metal M1 for example as a water soluble salt to a support in such a manner that the support remains dry in behaviour. The liquid is taken up into the pores of the support and preferably does not form a significant film on the outside of the catalyst. Subsequent removal of the solvent with, for example vacuum or nitrogen and/or heating leaves the catalyst precursor predominately in the pores.

For example, the metal M1 may be first loaded onto the substrate by an ion-exchange method, followed by the addition of the modifier.

Preferably after the addition of the metal M1 and the modifier to the acidic substrate, the acidic substrate is heat treated. The heat treatment may for example include heating to a temperature between from 450 to 800 degrees C., for example between from 500 to 600 degrees C.

The weight percent of metal M1 added to the acidic substrate in the method of preparation of the catalyst may be from about 0.1 wt % to about 2 wt %, for example from about 0.5 wt % to about 1 wt %.

The weight percent of metal M2 added to the acidic substrate relative to the acidic substrate, for example where M2 comprises Ca and the substrate comprises Y zeolite, is about from 0.1 wt % to about 2 wt %, for example from about 0.5 wt % to about 1 wt %.

The ratio of metal M2 to metal M1 by weight, for example where M2 comprises Ca and M1 comprises Pd, is between from about 0.1 to about 10, for example between from about 1 to 2.

In some examples, the method includes producing a hybrid catalyst, the method further including the step of mixing the modified catalyst and a carbon oxide(s) conversion catalyst, for example a methanol synthesis catalyst.

In preferred methods of preparing the hybrid catalyst, the modified catalyst is prepared initially and then is mixed with the carbon oxide(s) conversion catalyst.

The methanol synthesis catalyst may be any appropriate composition. In preferred examples, the catalyst includes Cu—ZnO-[Sup], Pd-[Sup] and Zn—Cr-[Sup], where [Sup] is preferably a support composition for example including Al₂O₃, SiO₂, and/or zeolite.

The hybrid catalyst may be prepared by any appropriate method, for example by a mechanical mixing method with methanol synthesis catalyst and modified zeolite. A granule mixing method may be used for example.

The weight percent of modified catalyst in the hybrid catalyst may be for example from about 20% to 80%, for example from about 40% to 70%.

Preferably the modified catalyst comprises a hydrogenation catalyst.

Preferably the hybrid catalyst is adapted for the conversion of carbon oxide(s) and hydrogen to form saturated hydrocarbons, in particular C₃ and higher saturated hydrocarbons.

Thus the invention further provides the use of a catalyst as described herein in the catalysed conversion of carbon oxide(s) and hydrogen to form saturated hydrocarbons.

According to a further aspect of the invention there is provided a process for the catalysed production of saturated hydrocarbons using a dehydration/hydrogenation catalyst including a modified catalyst, wherein the modified catalyst comprises:

an M1-zeolite or M1-SAPO catalyst, where M1 is a metal, and

a modifier including a metal M2, wherein M2 is an alkali metal or alkaline earth metal.

Preferably the modified catalyst is exposed to a source of a gas including methanol and/or DME and hydrogen.

The catalyst may comprise the modified catalyst and a further catalyst, for example a carbon oxide(s) conversion catalyst, for example a methanol synthesis catalyst.

The catalyst may comprise a hybrid catalyst as described herein.

The reactants may for example comprise syngas. Preferably the process includes feeding syngas to the dehydration/hydrogenation catalyst.

The process is preferably in gas phase. The reaction temperature may be between from about 260 to 400 degrees C., for example from about 290 to 335 degrees C. The reaction pressure may be between from about 0.5 to 6.0 MPa, for examples from 2.0 to 3.0 MPa. The gas space velocity may be from about 500 to 6000 h⁻¹, and for example about 1000 to 1500 h⁻¹. Preferably the gas space velocity is defined as the hourly volume of gas flow in standard units divided by the catalyst volume.

In some examples, the carbon oxide(s) conversion catalyst may be in a first stage which is separate from a second stage including the modified catalyst. In examples, the process may include an upstream catalyst bed including the carbon oxide(s) conversion catalyst, for example for the production of DME and/or methanol from carbon oxides and hydrogen. Thus the process may be carried out in a multiple stage system. For example a carbon oxide(s) conversion catalyst, for example a methanol synthesis catalyst may be provided in a first stage and the modified catalyst in a second stage. In some examples, the two stages will be separated. By separating the stages of the reaction system, it is possible to independently optimize the two stages. A significant advantage of this is that the methanol- and/or DME-generating catalyst can be run at conditions more suitable for improved conversion, selectivity, and/or longer catalyst life.

Preferably the first reaction stage temperature is lower than the second stage temperature, for example at least 20 degrees or at least 50 degrees lower. The temperature of the first stage may be less than 300 degrees C. Preferably, the temperature of the first stage is less than 295 degrees C., for example not more than 280 degrees C., for example not more than 250 degrees C. In examples, the temperature of the first stage may be between from about 190 to 250 degrees C., for example between from about 210 to 230 degrees C. In practical systems, it is likely that the temperature will vary across the reaction stage. Preferably the temperature of the stage is measured as an average temperature across a reaction region.

The temperature of the second stage may be more than 300 degrees C.

In some examples, the temperature of the second stage will be 320 degrees C. or more. In some examples, a temperature of 340 degrees C. or more will be preferred. In some examples the temperature of the second stage will be between from about 330 to 360 degrees C. In many cases it will be preferable for the temperature of the second stage to be less than 450 degrees C., for example less than 420 degrees C., or for example less than 400 degrees C. which may prolong the life of the catalyst. Depending on the target products, other temperatures may be used for the second stage.

The first and second stages may be operated at the same or at different pressures. Both stages may be operated for example at a pressure less than 40 bar. In some examples, it will be preferable for the second stage to be operated at a pressure lower than that of the first stage, for example at least 5 bar lower, for example at least 10 bar lower.

For example, the first stage may be operated at a pressure of less than 40 bar, less than 20 bar, or less than 10 bar. In some examples, a significantly higher pressure may be desirable.

For example, the second stage may be operated at a pressure of less than 20 bar, less than 10 bar, or less than 5 bar. In some examples, a significantly higher pressure may be desirable.

For LPG selectivity in the second stage, in some examples it will be preferable for the pressure of the second stage to be at least 1 MPa. In some examples it will be preferable for the pressure of the second stage to be less than about 2 MPa; in some examples, the selectivity of the process to methane is significant, which will be disadvantageous in many applications.

The gas hourly space velocity of the first stage may be for example between about 500 and 6000, for example between about 500 and 3000.

The gas hourly space velocity of the second stage may be for example between about 500 and 20000, for example between about 1000-10000.

Preferably the gas hourly space velocity is defined as the number of bed volumes of gas passing over the catalyst bed per hour at standard temperature and pressure.

Several configurations of the two stages are possible. An example giving less flexibility is one in which the two stages are contained within a single reactor vessel, for example as separate zones. In such a system, a heat transfer region may be provided, for example to control the reaction stage temperatures independently.

A more flexible system provides the two stages in separate vessels. At least a portion of the intermediate product stream (or effluent) exiting the first stage preferably passes directly to the second stage. Preferably, substantially all of the intermediate product stream passes to the second stage.

It will be understood that additional second stage influent components can be added to the intermediate stream upstream of the second stage. For example, addition of hydrogen and/or DME may be carried out. The intermediate stream may be subject to operations for example heat exchange upstream of the second stage and/or pressure adjustment, for example pressure reduction.

Each of the stages may include any appropriate catalyst bed type, for example fixed bed, fluidized bed, moving bed. The bed type of the first and second stages may be the same or different.

Potential application for example for the second stage is the use of a moving bed or paired bed system, for example a swing bed system, in particular where catalyst regeneration is desirable.

The feed to the process includes carbon oxide(s) and hydrogen. Any appropriate source of carbon oxides (for example carbon monoxide and/or carbon dioxide) and of hydrogen may be used. Processes for producing mixtures of carbon oxide(s) and hydrogen are well known. Each method has its advantages and disadvantages, and the choice of using a particular reforming process over another is normally governed by economic and available feed stream considerations, as well as by the desire to obtain the desired (H₂−CO₂):(CO+CO₂) molar ratio in the resulting gas mixture, that is suitable for further processing. Synthesis gas as used herein preferably refers to mixtures containing carbon dioxide and/or carbon monoxide with hydrogen. Synthesis gas may for example be a combination of hydrogen and carbon oxides produced in a synthesis gas plant from a carbon source such as natural gas, petroleum liquids, biomass and carbonaceous materials including coal, recycled plastics, municipal wastes, or any organic material. The synthesis gas may be prepared using any appropriate process for example partial oxidation of hydrocarbons (PDX), steam reforming (SR), advanced gas heated reforming (AGHR), microchannel reforming (as described in, for example, U.S. Pat. No. 6,284,217), plasma reforming, autothermal reforming (ATR) and any combination thereof.

A discussion of these synthesis gas production technologies is provided for in “Hydrocarbon Processing” V78, N.4, 87-90, 92-93 (April 1999) and/or “Petrole et Techniques”, N. 415, 86-93 (July-August 1998), which are both hereby incorporated by reference.

The synthesis gas source used in the present invention preferably contains a molar ratio of (H₂−CO₂):(CO+CO₂) ranging from 0.6 to 2.5. The gas composition which the catalyst is exposed to will generally differ from such a range due to for example gas recycling occurring within the reaction system. For example, in commercial methanol plants, a syngas feed molar ratio (as defined above) of 2:1 is commonly used, whereas the catalyst may experience a molar ratio of greater than 5:1 due to recycle. The gas composition experienced by the catalyst in the first stage where a two-stage process is used may initially be for example between from about 0.8 to 7, for example from about 2 to 3.

Carbon oxide(s) conversion catalysts for example methanol synthesis catalysts are commonly water gas shift active. The water gas shift reaction is the equilibrium of H₂ and CO₂ with CO and H₂O. The reaction conditions for the methanol synthesis catalyst (for example in the first stage) preferably favour the formation of H₂ and CO₂. For the case where the carbon oxide(s) conversion catalyst is active to produce methanol, the reaction stoichiometry requires a synthesis gas molar ratio of 2:1. For the case where the carbon oxides(s) conversion catalyst is active to produce dimethyl ether (DME), the reaction coproduces water which is shifted with CO according to the water gas shift reaction to CO₂ and hydrogen. In case, the synthesis gas molar ratio (as defined above) requirement is also 2:1 but here a reaction product is CO₂. The second part of the reaction, for example the second stage reaction in the case of methanol synthesis in the first stage is thought to comprise initial conversion to DME and water, and subsequent conversion of DME to C₃ and higher saturated hydrocarbons and water. The second stage reaction in the case of DME synthesis in the first stage is thought to comprise only the stages of DME conversion to C₃ and higher saturated hydrocarbons and water. In this case, the product mixture additionally includes carbon dioxide. Where a hybrid catalyst is used, these two stages will be in the same reactor.

The carbon oxides conversion catalyst preferably comprises a methanol conversion catalyst. The carbon oxides conversion catalyst may include Cu, or Cu and Zn. For example, the catalyst of the first stage may be based on a CuO/ZnO system. The catalyst may also include a support, for example alumina.

For the case where the carbon oxide(s) conversion catalyst is active to produce methanol, preferably no additional acid co-catalyst is present.

For the case where the carbon oxide(s) conversion catalyst is active to produce DME, an acid co-catalyst is preferably present. For example, the catalyst may include a zeolite and/or SAPO. This additional co-catalyst may also for example be used as a support for the methanol catalyst. Reference is made herein to a SAPO in addition to a zeolite. Preferably, where appropriate in the context, the term zeolite as used herein may also include SAPOs.

The carbon oxide(s) conversion catalyst may comprise a copper oxide. The catalyst may further include one or more metal oxides including Cu, Zn, Ce, Zr, Al, and Cr. For example, the carbon oxide(s) conversion catalyst may comprise Cu/Zn oxides for example on alumina. For example the catalyst may comprise CuO—ZnO—Al₂O₃.

The carbon oxide(s) conversion catalyst may include a zeolite and/or a SAPO, for example may include an acidic zeolite and/or a SAPO with stable structure like Mordenite, Y, ZSM-5, SAPO-11, SAPO-34.

The carbon oxide(s) conversion catalyst may comprise one or more of ZSM-5 and SAPO-11.

The content of carbon oxide(s) conversion catalyst in carbon oxide(s) conversion catalyst/M1-zeolite may be 20-80% (wt %), for example 30-60% (wt %), the percentage preferably being the ratio of the oxides to the zeolite, the measurement preferably being made for dry catalysts.

The hydrogenation catalyst may preferably include a metal, for example Pd.

The process may further include the step of carrying out a regeneration treatment of the modified catalyst. It is known that the MTO, MTP and MTG processes require frequent regeneration of the catalysts. One source of deactivation is the build up of coke formed on the catalysts during the reaction. While some of the modified catalysts of the present invention have greater resistance to coking, coke may nevertheless form on the catalyst. One way of removing such coke build up is by a controlled combustion method. Other methods include washing of the catalyst to remove the coke using for example aromatic solvent.

The regeneration of the catalyst may include heating the catalyst to a temperature of at least 500 degrees C. The temperature of the regeneration treatment may be for example at least 500 degrees C., preferably at least 550 degrees C., for example 580 degrees C. or more. It will be understood that a high temperature of treatment will be desirable to burn off the coke, but that very high temperatures will not be preferred in some cases because of the risk of reducing significantly the performance of the catalyst, for example due to metal sintering and/or zeolite thermal stability problems.

The regeneration of the modified catalyst may have added complexity where a metal is present in the catalyst as this can be affected adversely during the regeneration process. For example, the metal may sinter if a high temperature method is used. However, such sintered metals can be redispersed by an appropriate method such as treatment with carbon monoxide.

Where a hybrid catalyst is used, the hybrid catalyst may be subject to the regeneration treatment. Alternatively, the hybrid catalyst may first be separated, for example to separate the modified catalyst from any additional catalysts for example the carbon oxide(s) conversion catalyst, the regeneration treatment being carried out on the modified catalyst before adding fresh (or re-adding used or regenerated) carbon oxide(s) conversion catalyst.

During the regeneration treatment, some of the alkali or alkali earth (M2) metal may be lost. It is thought that this is particularly likely where a Group I metal is used as M2. Lost metal may be re-added to the catalyst after the regeneration treatment.

Thus in examples, the modified catalyst could be used repeatedly after regeneration by coke burning.

Preferably the product includes C₃ and/or higher saturated hydrocarbons. Thus aspects of the invention provide a method for producing C₃ and higher saturated hydrocarbons.

The product hydrocarbons preferably include iso-butane, wherein the proportion of iso-butane is preferably more than 60% by weight of the C₄ saturated hydrocarbons in the product. The C₄ fraction and higher hydrocarbons produced is preferably has a high degree of branching. This can be beneficial for applications in LPG, for example giving a reduced boiling point of the C₄ fraction, and/or for C₅ and higher hydrocarbons for octane number in gasoline. In addition, the use the product LPG including propane and iso-butane as a chemical feedstock to generate the corresponding olefins is preferable in some cases to using propane and n-butane. While examples of the invention have been described herein relating to the production of LPG, in other examples, target hydrocarbons include butane (C₄) and higher (C₅₊) hydrocarbons.

Many known syngas conversion processes are disadvantageous due to a low selectivity for the target product. One by-product which acts as a significant hydrogen sink is methane. The formation of methane can have a negative effect on the economics of the process. For example, Fischer Tropsch chemistry to produce diesel and alkanes typically produces more than 10% methane.

Preferably the molar fraction of methane in the total saturated hydrocarbons produced is less than 10%. Preferably the molar fraction of ethane in the total saturated hydrocarbons produced is less than 25%.

A further aspect of the invention provides an apparatus for carrying out a method as defined herein.

Also provided by the invention is apparatus for use in a process as described herein and a modified catalyst obtained or obtainable by a method described herein.

Aspects of the invention are applicable to other dehydration catalysts. A further aspect of the invention provides a modified catalyst for use as a dehydration/hydrogenation catalyst in a system for the catalysed production of saturated hydrocarbons from carbon oxides and hydrogen, the modified catalyst comprising:

an M1-[Sup], where M1 is a metal and [Sup] is an acidic support; and

a modifier including a metal M2,

wherein M2 is an alkali metal or alkaline earth metal.

Aspects of the invention may be applicable to microporous compositions, including for example to microporous compositions other than zeolites and SAPOs. For example, the modified catalyst may include metal organosilicates, silicalites and/or crystalline aluminophosphates.

For example, the acidic support may include a molecular sieve, or a crystalline microporous material. The catalyst may include a zeolite and/or a silicoalumino phosphate (SAPO), for example a crystalline microporous silicoalumino phosphate composition.

These features may be applied to any aspect of the invention.

In examples of aspects of the invention, hybrid catalyst has been found to give >70% carbon oxide(s) conversion and >70% LPG selectivity in hydrocarbons during 100 h reaction time, and to show good performance in the life test.

In examples, it has been identified that the presence, for example by addition, of M2 to the catalyst improves catalyst lifetime.

In examples of the invention, the stability of a hybrid catalyst for LPG synthesis from syngas has been improved by modifying one of its components. In examples, the hybrid catalyst includes methanol synthesis catalyst and a Pd-modified Y zeolite (Pd—Y). The presence of a metal, for example by the addition of Ca into the Pd—Y system has been found to hinder coke deposition on the Y zeolite, and thus improve the stability of hybrid catalyst.

The invention extends to a catalyst, method of preparing a catalyst system and/or use of a catalyst as herein described, preferably with reference to the accompanying drawings.

Any feature in one aspect of the invention may be applied to other aspects of the invention, in any appropriate combination. In particular, features of method aspects may be applied to apparatus and composition aspects, and vice versa.

Preferred features of aspects of the present invention will now be described, purely by way of example, with reference to the accompanying drawings, in which:

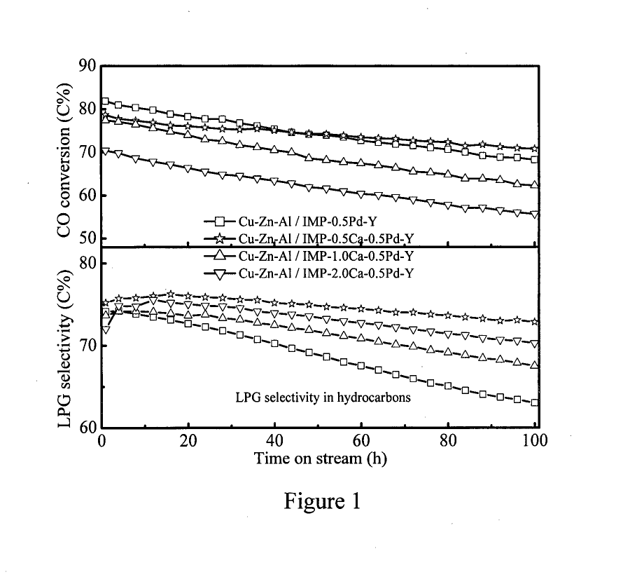

FIG. 1 is a graph of CO conversion and LPG selectivity in hydrocarbons for three example catalyst systems and one comparative example catalyst system;

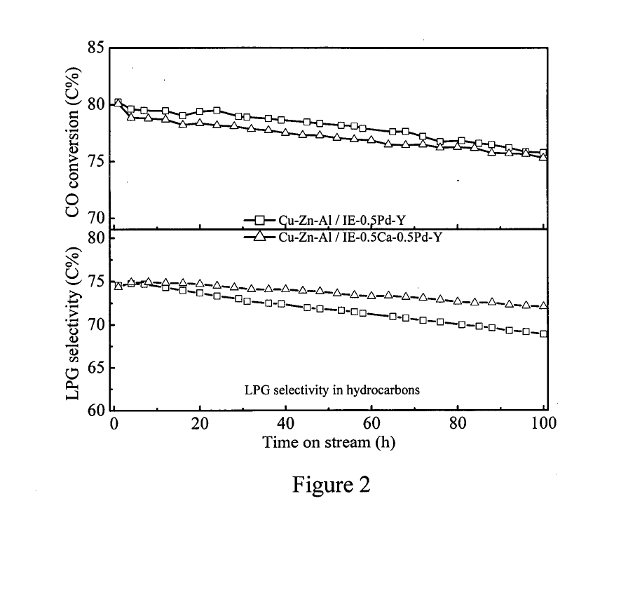

FIG. 2 is a graph of CO conversion and LPG selectivity in hydrocarbons for a further example catalyst system and a comparative example catalyst system;

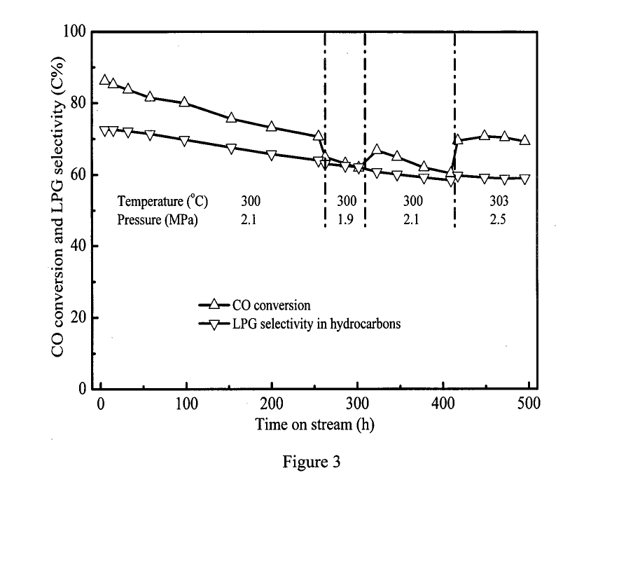

FIG. 3 is a graph of CO conversion and LPG selectivity in hydrocarbons for an example catalyst system under different pressure and temperature conditions;

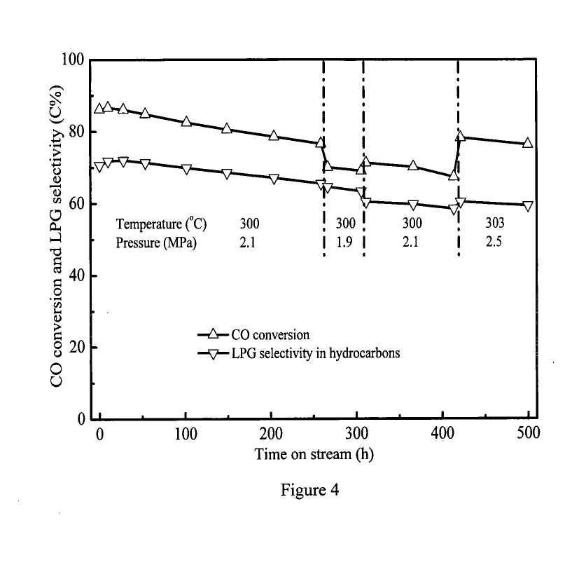

FIG. 4 is a graph of CO conversion and LPG selectivity in hydrocarbons for a further example catalyst system under different pressure and temperature conditions;

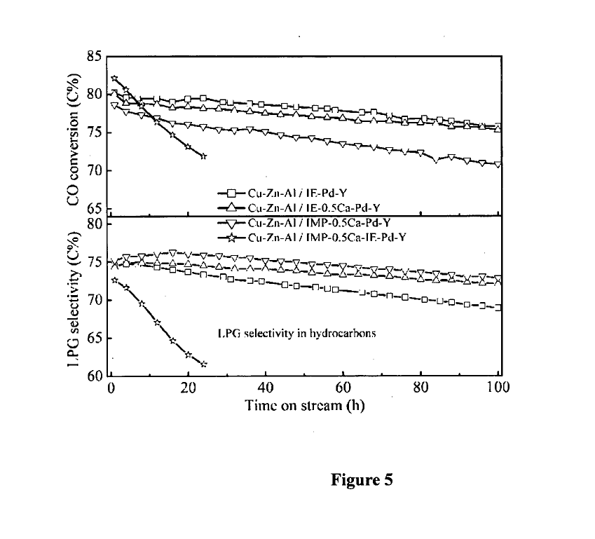

FIG. 5 is a graph of CO conversion and LPG selectivity in hydrocarbons for catalyst systems prepared using different methods;

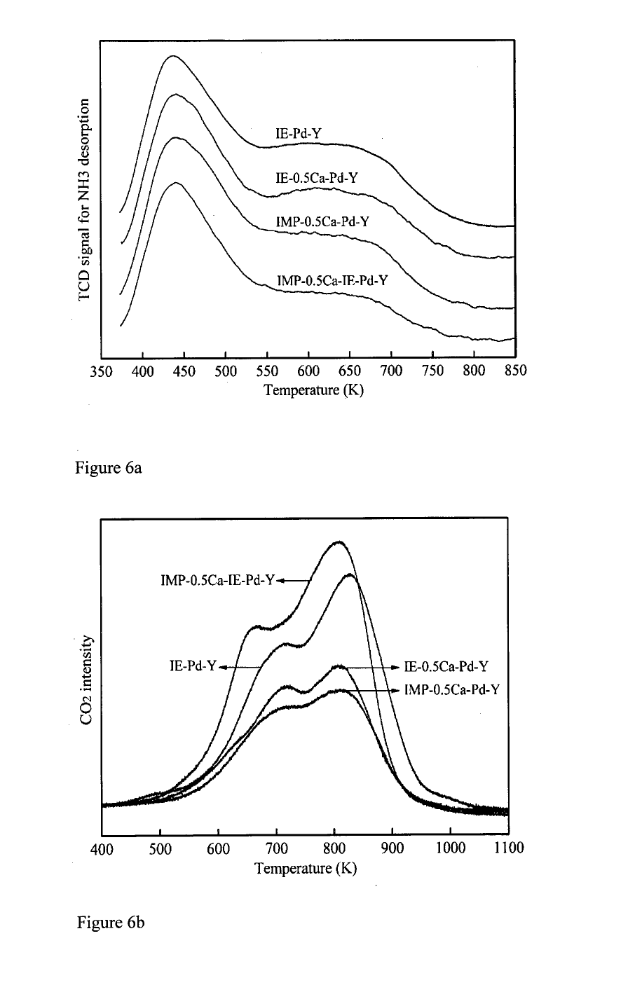

FIG. 6a shows NH₃-TPD profiles of modified Y zeolite catalyst before reaction;

FIG. 6b shows TPO-MS profiles of modified Y zeolite catalysts after reaction; and

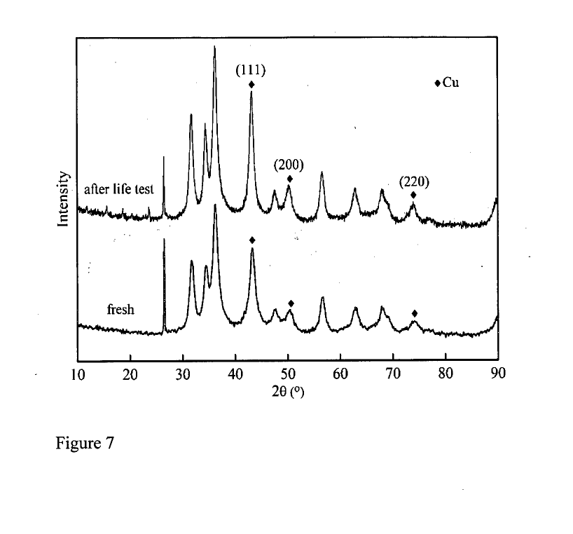

FIG. 7 shows XRD spectra of a methanol synthesis catalyst.

Example catalysts and methods for their preparation and evaluation are now described. In the examples, a modified dehydration/hydrogenation catalysts are formed from Y zeolite is and the addition of Pd and Ca, and the modified catalysts are used as a component of a hybrid catalyst for use in the production of LPG from syngas. The methods used for the preparation of the modified catalyst for these examples included an ion-exchange method and incipient-wetness impregnation method. The Y zeolite used in these experiments was a proton-typed zeolite obtained from Nankai University Catalyst Ltd. The Y-zeolite was Na-typed after synthesis and was treated by Nankai University Catalyst Ltd by an ion-exchange method with ammonium salt (for example NH₄NO₃) followed by calcination to obtain the proton-typed Y-zeolite. The ratio of silica to alumina in the Y-zeolite was 6.

The modified catalysts were mixed with a methanol synthesis catalyst to form hybrid catalysts and the hybrid catalysts were used in a process for the production of saturated hydrocarbons from syngas. The production of saturated hydrocarbons from syngas over the hybrid catalysts comprising methanol synthesis catalyst and modified zeolite is believed to involve the following steps: CO hydrogenation to form methanol over the methanol synthesis catalyst; methanol dehydration to form DME, and further dehydration to form olefins over the zeolite, and olefins hydrogenation to form saturated hydrocarbons over the active metal supported on the zeolite.

In the evaluation experiments a pressurized flow type reaction apparatus with a fixed bed reactor was used. The apparatus was equipped with an electronic temperature controller for a furnace, a tubular reactor with an inner diameter of 10 mm, thermal mass flow controllers for gas flows and a back-pressure valve. The catalyst used in the reactor was activated at 250 degrees C. for 5 hours in a pure hydrogen flow. The feed was introduced into the reactor in gaseous state and products were analysed by gas chromatography (GC) on line. The presence of CO, CO₂, CH₄ and N₂ were analysed using a GC apparatus equipped with a thermal conductivity detector (TCD), and the presence of organic compounds were analysed by another GC apparatus equipped with a flame ionization detector (FID).

Catalyst characterisation was carried out using temperature programmed oxidation and mass spectrometric detection (TPO-MS) using a quadrupole mass spectrometer GSD 301 (Pfeiffer). A 60 mg sample was heated from ambient temperature to 900 degrees C. with a heating rate of 10 degrees C./min under a flow of 5% O₂ and 95% Ar. Temperature-programmed desorption of NH₃ (NH₃-TPD) was conducted on the Autochem 2910 apparatus (Micromeritics). A 100 mg sample was heated from 100 to 700 degrees C. at a constant rate of 10 degrees C./min after saturation sorption of NH₃.

### Example 1

0.5 wt % Pd and 0.5 wt % Ca relative to the weight of the zeolite were loaded into a Y zeolite simultaneously by incipient-wetness impregnation method. PdCl₂ and Ca(NO₃)₂.4H₂O as the precursors of Pd and Ca, respectively, were dissolved in water. About 9 ml solution was dropped onto 10 g of Y zeolite in 5 min, maintained for 12 h at room temperature, and then dried at 120 degrees C. and calcined at 550 degrees C. for 6 h. The resulting product is denoted herein as IMP-0.5Ca-0.5Pd—Y.

A methanol synthesis catalyst comprising CuO and ZnO on an Al₂O₃ support (a commercial methanol synthesis catalyst from Shenyang Catalyst Corp. was used) was granule mixed with IMP-0.5Ca-0.5Pd—Y at a weight ratio of 7:9. The hybrid catalyst formed is denoted herein as Cu—Zn—Al/IMP-0.5Ca-0.5Pd—Y.

### Example 2

0.5 wt % Pd and 1.0 wt % Ca were loaded into a Y zeolite simultaneously by an incipient-wetness impregnation method similar to that as described in relation to Example 1. The resulting product is denoted herein as IMP-1.0Ca-0.5Pd—Y.

A methanol synthesis catalyst as described in Example 1 was granule mixed with IMP-1.0Ca-0.5Pd—Y at a weight ratio of 7:9. The hybrid catalyst formed is denoted herein as Cu—Zn—Al/IMP-1.0Ca-0.5Pd—Y.

### Example 3

0.5 wt % Pd and 2.0 wt % Ca were loaded into a Y zeolite simultaneously by incipient-wetness impregnation method similar to that described in relation to Example 1. The resulting product is denoted herein as IMP-2.0Ca-0.5Pd—Y.

A methanol synthesis catalyst as described in Example 1 was granule mixed with IMP-2.0Ca-0.5Pd—Y at a weight ratio of 7:9. The hybrid catalyst formed is denoted herein as Cu—Zn—Al/IMP-2.0Ca-0.5Pd—Y.

### Example 4

0.5 wt % Pd and 0.5 wt % Ca were loaded into a Y zeolite simultaneously by an ion-exchange method. 10 g Y zeolite was added to a 200 ml agitated solution of PdCl₂ and Ca(NO₃)₂.4H₂O at 60 degrees C., and maintained for 8 h, and then washed with water, dried at 120 degrees C. and calcined at 550 degrees C. for 6 h. The resulting product is denoted herein as IE-0.5Ca-0.5Pd—Y.

A methanol synthesis catalyst as described in Example 1 was granule mixed with IE-0.5Ca-0.5Pd—Y at a weight ratio of 7:9. The hybrid catalyst formed is denoted herein as Cu—Zn—Al/IE-0.5Ca-0.5Pd—Y.

### Comparative Example 1

0.5 wt % Pd was loaded into a Y zeolite by an incipient-wetness impregnation method. The PdCl₂ as the precursors Pd were dissolved in water. About 9 ml solution was dropped to 10 g Y zeolite in 5 min, maintained for 12 h at room temperature, and then dried at 120 degrees C. and calcined at 550 degrees C. for 6 h. The resulting product is denoted herein as IMP-0.5Pd—Y.

A methanol synthesis catalyst as described in Example 1 was granule mixed with IMP-0.5Pd—Y at a weight ratio of 7:9. The hybrid catalyst formed is denoted herein as Cu—Zn—Al/IMP-0.5Pd—Y.

### Comparative Example 2

0.5 wt % Pd was loaded into a Y zeolite by ion-exchange method. 10 g Y zeolite was added to a 200 ml agitated solution of PdCl₂ at 60 degrees C., and maintained for 8 h, and then washed with water, dried at 120 degrees C. and calcined at 550 degrees C. for 6 h. The resulting product is denoted herein as IE-0.5Pd—Y.

A methanol synthesis catalyst as described in Example 1 was granule mixed with IE-0.5Pd—Y at a weight ratio of 7:9. The hybrid catalyst formed is denoted herein as Cu—Zn—Al/IE-0.5Pd—Y.

### Experiment 1

The hybrid catalysts of Examples 1, 2 and 3 and of Comparative Example 1 were evaluated in a process for the reaction of syngas to form hydrocarbons including LPG. The feed gas comprised hydrogen, carbon monoxide and nitrogen at a weight ratio of H₂:CO:N₂ being 64:32:4. The pressure of the reaction was 2.1 MPa and the gas hourly space velocity was 1500. The reaction temperature was 290 degrees C. for all hybrid catalyst examples except for the catalyst of Example 3 for which the reaction temperature was 300 degrees C.

The higher reaction temperature of 300 degrees C. for the catalyst of Example 3 was selected in view of the increased Ca-content of the catalyst of Example 3 and the belief that therefore the amount of acid sites of the zeolite were decreased. In that case, it was considered that the higher temperature was appropriate for the desired conversion of most of any methanol and dimethyl ether formed in the reaction to form the desired hydrocarbon products.

The results are listed in Table 1 and shown in FIG. 1.

Besides transforming the syngas into hydrocarbons, about 46% CO was seen to be converted to CO₂ through the water-gas shift reaction. In addition, a trace amount of methanol and/or DME was also generated. The products of the process of the conversion of syngas to LPG therefore comprised CO₂, methanol, DME and hydrocarbons. The selectivity to CO₂ for all the hybrid catalysts was seen to be similar. Excluding the trace amount of methanol and/or DME, it was seen that the selectivity of hydrocarbons was in each case 54%. In order to clearly compare the performance of the various hybrid catalysts, only CO conversion and LPG selectivity in hydrocarbons were shown in some of the Tables herein.

Table 1 shows that both CO conversion and LPG selectivity in hydrocarbons over all the hybrid catalysts gradually decreased with time on stream. However, the rate of decrease was significantly different for the different catalysts. The decrease in the rate of CO conversion for the catalysts of Examples 2 and 3 and Comparative Example 1 was similar. The decrease in the rate of LPG selectivity in hydrocarbons for the catalysts of Example 2 and 3 was slower than that for the catalyst of Comparative Example 1. The decrease in the rate for both CO conversion and LPG selectivity in hydrocarbons for the catalyst of Example 1 was slower than that for the catalyst of Comparative Example 1.

TABLE 1
Effect of Ca promoter (impregnation method)

| | Hybrid catalyst | | | | | | | |
|---|---|---|---|---|---|---|---|---|
| | Cu—Zn—Al/ | | Cu—Zn—Al/ | | Cu—Zn—Al/ | | Cu—Zn—Al/ | |
| | IMP- | | IMP- | | IMP- | | IMP- | |
| | IMP-0.5Pd—Y | | 0.5Ca—0.5Pd—Y | | 1.0Ca—0.5Pd—Y | | 2.0Ca—0.5Pd—Y | |
| | CO | LPG | CO | LPG | CO | LPG | CO | LPG |
| Time | conversion | selectivity | conversion | selectivity | conversion | selectivity | conversion | selectivity |
| (h) | (C %) | (C %) | (C %) | (C %) | (C %) | (C %) | (C %) | (C %) |
| 1 | 81.88 | 74.15 | 78.63 | 75.2 | 77.42 | 73.67 | 70.43 | 72.04 |
| 4 | 80.98 | 74.22 | 77.77 | 75.73 | 77.09 | 74.17 | 69.81 | 74.84 |
| 8 | 80.37 | 73.86 | 77.33 | 75.81 | 76.48 | 74.17 | 68.66 | 74.77 |
| 12 | 79.8 | 73.5 | 76.9 | 76.04 | 75.6 | 74.11 | 67.88 | 75.57 |
| 16 | 78.88 | 73.13 | 76.25 | 76.26 | 74.8 | 73.9 | 67.19 | 75.22 |
| 20 | 78.25 | 72.66 | 76.06 | 76.04 | 74.02 | 73.65 | 66.37 | 75.05 |
| 24 | 77.74 | 72.28 | 75.77 | 75.9 | 72.97 | 73.76 | 65.53 | 74.87 |
| 28 | 77.67 | 71.81 | 75.4 | 75.81 | 72.6 | 73.33 | 64.84 | 74.77 |
| 32 | 76.77 | 71.29 | 75.27 | 75.59 | 71.65 | 73.14 | 64.53 | 74.56 |
| 36 | 76.1 | 70.75 | 75.46 | 75.52 | 71.14 | 72.77 | 63.92 | 74.16 |
| 40 | 75.38 | 70.25 | 75.09 | 75.18 | 70.48 | 72.49 | 63.43 | 73.95 |
| 44 | 74.6 | 69.64 | 74.65 | 75.08 | 69.95 | 72.14 | 62.8 | 73.76 |
| 48 | 74.17 | 69.15 | 74.34 | 74.97 | 68.6 | 71.88 | 61.94 | 73.57 |
| 52 | 73.93 | 68.63 | 74.26 | 74.7 | 68.18 | 71.48 | 61.59 | 73.24 |
| 56 | 73.51 | 68.01 | 73.86 | 74.6 | 67.68 | 71.17 | 60.95 | 72.97 |
| 60 | 72.71 | 67.54 | 73.51 | 74.46 | 67.41 | 70.85 | 60.37 | 72.74 |
| 64 | 72.26 | 67.02 | 73.27 | 74.27 | 66.94 | 70.46 | 60.08 | 72.5 |
| 68 | 71.87 | 66.48 | 73.1 | 74.01 | 66.38 | 70.11 | 59.62 | 72.21 |
| 72 | 71.52 | 65.98 | 72.7 | 74.02 | 65.52 | 69.85 | 59.05 | 71.97 |
| 76 | 71.04 | 65.44 | 72.5 | 73.79 | 65.3 | 69.43 | 58.42 | 71.7 |
| 80 | 70.68 | 65.07 | 72.35 | 73.62 | 64.83 | 69.15 | 57.83 | 71.42 |
| 84 | 70.02 | 64.53 | 71.43 | 73.44 | 63.9 | 68.82 | 57.08 | 71.25 |
| 88 | 69.19 | 64.08 | 71.81 | 73.27 | 63.85 | 68.47 | 57.11 | 70.91 |
| 92 | 68.83 | 63.72 | 71.28 | 72.98 | 63.51 | 68.26 | 56.51 | 70.68 |
| 96 | 68.73 | 63.43 | 70.98 | 73.07 | 62.53 | 67.9 | 56.02 | 70.56 |
| 100 | 68.27 | 63.02 | 70.79 | 72.85 | 62.29 | 67.52 | 55.69 | 70.27 |

Note:
LPG selectivity in this example means LPG selectivity in hydrocarbons

Therefore, it was seen that the hybrid catalyst with Ca exhibited higher stability than the hybrid catalyst without Ca, especially in relation to the LPG selectivity in hydrocarbons. This suggests that the introduction of Ca for example by an incipient-wetness impregnation method benefits the stability of the hybrid catalyst, for example in a process for LPG synthesis from syngas.

As discussed above, without wishing to be bound by any particular theory, it is believed that increasing concentration of Ca in solution would lead to the decrease of Pd content and increase of Ca content in the modified Y zeolite. NH₃-TPD profiles suggested that the increase of Ca content resulted in the gradual decrease of the strong acid sites of modified Y zeolite. It is thought that the decrease of the strong acid sites would suppress the formation of coke and improve the stability of the hybrid catalyst. However, the decrease of Pd content is believed to reduce the hydrogenation ability of the hybrid catalyst so that more coke forms during the same reaction period. Thus it is believed that there is a conflict between the advantage obtained from the increase in Ca, and the disadvantage of the Pd decrease. Thus it was seen for this example that the stability of the hybrid catalyst first improved with increasing Ca content, then got worse. In this example, a preferred ratio was 0.5% of Ca relative to the Y zeolite in the ion-exchange solution.

### Experiment 2

The catalysts of Example 4 and of Comparative Example 2 were evaluated in a process for the reaction of syngas to form hydrocarbons including LPG. The reaction temperature was 290 degrees C., the reaction pressure was 2.1 MPa, and the gas hourly space velocity was 1500. The feed gas included hydrogen, carbon monoxide and nitrogen at a ratio of H₂:CO:N₂ of 64:32:4. The results are listed in Table 2 and shown in FIG. 2.

TABLE 2
Effect of Ca promoter (ion-exchange method)

| Cu—Zn—Al/IMP-0.5Pd—Y | | | Cu—Zn—Al/IMP-0.5Ca—0.5Pd—Y | | |
|---|---|---|---|---|---|
| | CO | LPG | | CO | LPG |
| Time | conversion | selectivity | Time | conversion | selectivity |
| (h) | (C %) | (C %) | (h) | (C %) | (C %) |
| 1 | 80.23 | 74.57 | 1 | 80.08 | 74.36 |
| 4 | 79.61 | 74.75 | 4 | 78.85 | 74.91 |
| 7 | 79.48 | 74.69 | 8 | 78.79 | 74.93 |
| 12 | 79.45 | 74.32 | 12 | 78.70 | 74.85 |
| 16 | 79.04 | 73.98 | 16 | 78.23 | 74.8 |
| 20 | 79.39 | 73.67 | 20 | 78.37 | 74.72 |
| 24 | 79.49 | 73.32 | 24 | 78.17 | 74.50 |
| 29 | 78.96 | 73.02 | 28 | 78.09 | 74.30 |
| 31 | 78.91 | 72.71 | 32 | 77.86 | 74.10 |
| 36 | 78.78 | 72.48 | 36 | 77.74 | 74.07 |
| 39 | 78.64 | 72.41 | 40 | 77.50 | 74.08 |
| 45 | 78.46 | 71.97 | 44 | 77.33 | 73.91 |
| 48 | 78.34 | 71.81 | 48 | 77.29 | 73.85 |
| 53 | 78.17 | 71.65 | 52 | 77.06 | 73.59 |
| 56 | 78.11 | 71.47 | 56 | 76.93 | 73.39 |
| 58 | 77.91 | 71.30 | 60 | 76.85 | 73.28 |
| 65 | 77.60 | 70.93 | 64 | 76.48 | 73.34 |
| 68 | 77.63 | 70.73 | 68 | 76.44 | 73.19 |
| 72 | 77.20 | 70.48 | 72 | 76.48 | 73.08 |
| 76 | 76.75 | 70.28 | 76 | 76.21 | 72.88 |
| 81 | 76.83 | 69.98 | 80 | 76.26 | 72.63 |
| 85 | 76.60 | 69.78 | 84 | 76.18 | 72.51 |
| 88 | 76.46 | 69.61 | 88 | 75.74 | 72.56 |
| 92 | 76.19 | 69.29 | 92 | 75.70 | 72.28 |
| 96 | 75.84 | 69.15 | 96 | 75.65 | 72.17 |
| 100 | 75.77 | 68.86 | 100 | 75.30 | 72.08 |

Note:
LPG selectivity in this example means LPG selectivity in hydrocarbons

The decreases in the rate of CO conversion for the catalysts of Example 4 and Comparative Example 2 were similar. However, the decrease in the rate of LPG selectivity in hydrocarbons for the catalyst of Example 4 was slower than that for the catalyst of the Comparative Example 2.

It was therefore identified that in this example, the introduction of Ca for example by ion-exchange method also benefited the stability of hybrid catalyst, for example in relation to the production of LPG from syngas.

### Experiment 3

A life test of the catalyst of Example 1 was carried out in a process for the reaction of syngas to form hydrocarbons including LPG. In this example, the feed gas included hydrogen, carbon monoxide and nitrogen at a ratio of H₂:CO:N₂ being 64:32:4. The gas hourly space velocity was 1100. The temperature and pressure were modulated several times during the process of reaction as indicated in Table 3. The representative results are listed in Table 3 and are shown in FIG. 3.

Both CO conversion and LPG selectivity for the catalyst of Example 1 were seen to gradually decrease with time on stream when the experiment conditions were not altered. The increase of reaction pressure was seen to benefit both CO conversion and LPG selectivity each time. However, the increase in reaction pressure in this example did not change the overall download trend in the CO conversion and LPG selectivity. LPG selectivity in hydrocarbons became relatively stable in the later stage of the reaction in this example.

TABLE 3
Life test of hybrid catalyst (impregnation method)
Cu—Zn—Al/IMP-0.5Ca—0.5Pd—Y

| | | | CO | LPG selectivity |
|---|---|---|---|---|
| Time | Temperature | Pressure | conversion | in hydrocarbons |
| (h) | (° C.) | (MPa) | (C %) | (C %) |
| 5.27 | 300 | 2.1 | 86.21 | 72.61 |
| 15.05 | 300 | 2.1 | 85.22 | 72.57 |
| 32.49 | 300 | 2.1 | 83.73 | 72.18 |
| 57.95 | 300 | 2.1 | 81.50 | 71.41 |
| 98.17 | 300 | 2.1 | 78.98 | 69.76 |
| 153.05 | 300 | 2.1 | 75.64 | 67.61 |
| 200.09 | 300 | 2.1 | 73.22 | 65.83 |
| 254.98 | 300 | 2.1 | 70.63 | 63.99 |
| 262.82 | 300 | 1.9 | 64.85 | 62.96 |
| 286.33 | 300 | 1.9 | 63.06 | 62.48 |
| 302.02 | 300 | 1.9 | 61.94 | 62.08 |
| 323.21 | 300 | 2.1 | 66.82 | 60.77 |
| 346.74 | 300 | 2.1 | 64.86 | 60.13 |
| 378.1 | 300 | 2.1 | 62.01 | 59.19 |
| 409.46 | 300 | 2.1 | 60.44 | 58.36 |
| 417.3 | 303 | 2.5 | 69.53 | 59.79 |
| 448.67 | 303 | 2.5 | 70.73 | 59.18 |
| 472.19 | 303 | 2.5 | 70.35 | 58.92 |
| 495.72 | 303 | 2.5 | 69.34 | 58.99 |

### Experiment 4

The two components of the hybrid catalyst were separated from each other after the Experiment 3 (more than 700 hours). The deactivated IMP-0.5Ca-0.5Pd—Y was regenerated using a regeneration treatment.

The regeneration treatment in this experiment included coke burning in a 5% O₂, 95% Ar gaseous mixture until no CO₂ was detected. The detection was carried out downstream of the coke burning using a thermal conductivity detector (TCD). In this example, the temperature of the regeneration treatment was 580 degrees C.

After the regeneration treatment, the catalyst was mixed with fresh methanol synthesis catalyst having a similar composition and using the method of mixing as described in Example 1. The regenerated catalyst was returned to the apparatus and the reaction continued to convert syngas to LPG at a reaction temperature of 290 degrees C., pressure of 2.1 MPa, GHSV of 1500 and using a feed gas comprising hydrogen, carbon monoxide an nitrogen in a ratio H₂:CO:N₂ of 64:32:4. The results are listed in Table 4.

It was seen that the decrease in performance, thought to be a result of an effect of coke deposition, could be removed to a significant extent by using coke burning in the regeneration treatment. It was found that the modified zeolite could be used repeatedly after regeneration.

TABLE 4
Comparison between fresh and regenerated
catalyst (impregnation method)

| | | CO | LPG selectivity |
|---|---|---|---|
| Hybrid catalyst | | conversion | in hydrocarbons |
| Cu—Zn—Al | IMP-0.5Ca—0.5Pd—Y | (C %) | (C %) |
| Fresh | fresh | 79.24 | 73.50 |
| Fresh | deactivated | 71.08 | 56.38 |
| Fresh | regenerated | 78.10 | 72.70 |

### Experiment 5

A life test using the catalyst of Example 4 was carried out using a feed gas having hydrogen, carbon monoxide and nitrogen in a ratio of H₂:CO:N₂ of 64:32:4. The gas space velocity was 1100 h⁻¹. The temperature and pressure were modulated several times during the process of reaction. The representative results are listed in Table 5a and Table 5b and are shown in FIG. 4.

The trend in performance for the catalyst of Example 4 was seen to be similar to that of the catalyst of Example 1. This suggested that the introduction of Ca to the catalyst by the incipient-wetness impregnation method or ion-exchange method have a similar effect on the stability of the hybrid catalyst in these examples.

TABLE 5a
Life test of hybrid catalyst (ion-exchange method)
Cu—Zn—Al/IE-0.5Ca—0.5Pd—Y

| | | | CO | LPG selectivity |
|---|---|---|---|---|
| Time | Temperature | Pressure | conversion | in hydrocarbons |
| (h) | (° C.) | (MPa) | (C %) | (C %) |
| 0.66 | 300 | 2.1 | 86.15 | 70.59 |
| 10.54 | 300 | 2.1 | 86.65 | 71.84 |
| 28.58 | 300 | 2.1 | 86.1 | 72.03 |
| 54.04 | 300 | 2.1 | 84.81 | 71.36 |
| 102.39 | 300 | 2.1 | 82.53 | 69.89 |
| 149.44 | 300 | 2.1 | 80.60 | 68.61 |
| 204.32 | 300 | 2.1 | 78.57 | 67.15 |
| 259.2 | 300 | 2.1 | 76.63 | 65.50 |
| 267.04 | 300 | 1.9 | 70.04 | 64.60 |
| 290.56 | 300 | 1.9 | 69.02 | 63.71 |
| 306.24 | 300 | 1.9 | 69.06 | 63.35 |
| 311.76 | 300 | 2.1 | 71.29 | 60.41 |
| 366.64 | 300 | 2.1 | 70.16 | 59.80 |
| 390.16 | 300 | 2.1 | 68.76 | 59.23 |
| 413.69 | 300 | 2.1 | 67.40 | 58.52 |
| 421.53 | 303 | 2.5 | 78.35 | 60.41 |
| 437.21 | 303 | 2.5 | 78.49 | 59.78 |
| 476.41 | 303 | 2.5 | 76.85 | 58.79 |
| 499.94 | 303 | 2.5 | 76.34 | 59.35 |

TABLE 5b
Life test of hybrid catalyst (ion-exchange method)

| | | | CO | | | | | | | |
|---|---|---|---|---|---|---|---|---|---|---|
| Time | Temperature | Pressure | conversion | Hydrocarbon distribution (C %) | | | | | | |
| (h) | (° C.) | (MPa) | (C %) | C₁ | C₂ | C₃ | C₄ | C₅ | C₆₊ | LPG |
| 0.66 | 300 | 2.1 | 86.15 | 7.75 | 11.49 | 21.40 | 49.19 | 8.73 | 1.44 | 70.59 |
| 10.54 | 300 | 2.1 | 86.65 | 6.78 | 12.94 | 25.76 | 46.07 | 7.13 | 1.32 | 71.84 |
| 28.58 | 300 | 2.1 | 86.10 | 5.77 | 13.26 | 24.36 | 47.67 | 7.66 | 1.28 | 72.03 |
| 54.04 | 300 | 2.1 | 84.81 | 5.34 | 13.66 | 23.32 | 48.05 | 8.24 | 1.39 | 71.36 |
| 102.39 | 300 | 2.1 | 82.53 | 5.20 | 14.25 | 22.25 | 47.65 | 9.10 | 1.55 | 69.89 |
| 149.44 | 300 | 2.1 | 80.60 | 5.41 | 14.54 | 21.57 | 47.04 | 9.76 | 1.68 | 68.61 |
| 204.32 | 300 | 2.1 | 78.57 | 5.66 | 14.74 | 20.77 | 46.38 | 10.62 | 1.83 | 67.15 |
| 259.20 | 300 | 2.1 | 76.63 | 6.03 | 14.94 | 19.92 | 45.58 | 11.44 | 2.09 | 65.50 |
| 267.04 | 300 | 1.9 | 70.04 | 6.73 | 15.26 | 19.89 | 44.71 | 11.45 | 1.96 | 64.60 |
| 290.56 | 300 | 1.9 | 69.02 | 6.66 | 15.25 | 19.45 | 44.47 | 12.10 | 2.07 | 63.71 |
| 306.24 | 300 | 1.9 | 69.06 | 6.62 | 15.32 | 19.20 | 44.14 | 12.37 | 2.35 | 63.35 |
| 311.76 | 300 | 2.1 | 71.29 | 6.22 | 15.17 | 17.55 | 42.86 | 15.46 | 2.74 | 60.41 |
| 366.64 | 300 | 2.1 | 70.16 | 7.52 | 15.44 | 17.73 | 42.08 | 14.66 | 2.57 | 59.80 |
| 390.16 | 300 | 2.1 | 68.76 | 7.77 | 15.39 | 17.47 | 41.76 | 14.99 | 2.62 | 59.23 |
| 413.69 | 300 | 2.1 | 67.40 | 8.17 | 15.27 | 17.13 | 41.39 | 15.34 | 2.7 | 58.52 |
| 421.53 | 303 | 2.5 | 78.35 | 7.62 | 14.61 | 17.31 | 43.11 | 14.72 | 2.63 | 60.41 |
| 437.21 | 303 | 2.5 | 78.49 | 8.59 | 14.59 | 17.23 | 42.55 | 14.49 | 2.55 | 59.78 |
| 476.41 | 303 | 2.5 | 76.85 | 9.89 | 14.30 | 16.81 | 41.98 | 14.49 | 2.53 | 58.79 |
| 499.94 | 303 | 2.5 | 76.34 | 9.07 | 14.38 | 16.80 | 42.55 | 14.81 | 2.39 | 59.35 |

### Experimental 6

The two components of the hybrid catalyst were separated from each other after the Experiment 5 (over 700 hours). The deactivated IE-0.5Ca-0.5Pd—Y catalyst was regenerated by coke burning using a method as described in Experiment 4, and mixed with fresh methanol synthesis catalyst and the resulting hybrid catalyst was evaluated in relation to the reaction of syngas to form LPG at a reaction temperature of 290 degrees C., pressure of 2.1 MPa, and gas space velocity of 1500 h⁻¹ using a feed gas including hydrogen, carbon monoxide and nitrogen at a ratio of H₂:CO:N₂ of 64:32:4. The results are listed in Table 6. The effect of the regeneration treatment was seen to be similar to that in relation to Experiment 4.

The results in Table 6 suggested that the decrease of CO conversion may be mostly attributable to the deactivation of Cu in methanol synthesis catalyst and coke deposition on modified Y zeolite was the secondary factor. FIG. 7 shows the XRD spectra of the methanol synthesis catalyst before and after reaction. The characteristic peaks of Cu became clearer and narrower after the life test. This implied the increase of Cu particle size due to sintering. The increase of Cu particle size from 15.8 nm to 27.5 nm based on (111) reduced the effective surface area of Cu, and thus is thought to have decreased CO conversion.

Without wishing to be bound by any particular theory, the following is noted. As discussed earlier, olefins hydrogenation to form paraffins over the active metal supported on zeolite was one step in the process of saturated hydrocarbon synthesis from syngas. For the hybrid catalyst I, due to the low CO conversion, the amount of olefins generated by the active metal Pd was less than that over the fresh hybrid catalyst. It resulted in the increase of C₂, decrease of C₅₊ and a relatively low average weight of hydrocarbons. Nonetheless, LPG selectivity was similar to that over the fresh hybrid catalyst. Both CO conversion and hydrocarbon distribution over the hybrid catalyst III were very close to that over the fresh hybrid catalyst. It implied that the negative influence of coke deposition on the performance could be substantially eliminated by coke burning. For the hybrid catalyst II, LPG selectivity was much lower than that using the other three hybrid catalysts. The above results suggested that coke deposition was the main contribution to the decrease of LPG selectivity.

In addition, both C₂ and C₅₊ selectivity using the hybrid catalyst II were higher than that using the fresh hybrid catalyst. Coke deposition on modified Y zeolite was thought to impair the hydrogenation ability of IE-0.5Ca—Pd—Y. The polymerization of olefins could not be restrained effectively. Thus, a considerable number of C₅₊ hydrocarbons appeared. On the other hand, the high C₂ selectivity may have been caused by the change of pore size of Y zeolite.

TABLE 6
Comparison between fresh and regenerated catalyst (ion-exchange method)

| | | | CO | | | | | | | |
|---|---|---|---|---|---|---|---|---|---|---|
| | Hybrid catalyst | | conversion | Hydrocarbon distribution (C %) | | | | | | |
| No. | Cu—Zn—Al | IE-0.5Ca—Pd—Y | (C %) | C₁ | C₂ | C₃ | C₄ | C₅ | C₆₊ | LPG |
| fresh | fresh | fresh | 80.7 | 5.1 | 9.8 | 26.4 | 48.0 | 8.1 | 2.6 | 74.5 |
| I | deactivated | fresh | 55.3 | 3.5 | 13.8 | 32.8 | 42.3 | 5.9 | 1.7 | 75.1 |
| II | fresh | deactivated | 71.8 | 8.4 | 12.7 | 15.7 | 39.1 | 17.0 | 7.1 | 54.8 |
| III | fresh | regenerated | 79.7 | 4.9 | 9.7 | 23.6 | 50 | 9.0 | 2.8 | 73.6 |

### Experiment 7

Hybrid catalysts were prepared using different methods including incipient-wetness impregnation (IMP) and ion exchange (IE) equivalent to methods described above to make hybrid catalysts as follows:

### Cu—Zn—Al/IE-Pd—Y

### Cu—Zn—Al/IE-0.5Ca—Pd—Y

### Cu—Zn—Al/IMP-0.5Ca—Pd—Y

### Cu—Zn—Al/IMP-0.5Ca-IE-Pd—Y

For the first three catalysts, the Pd and Ca were added together to the Y zeolite. In the fourth catalyst, the Ca was first added by IMP, followed by the addition of Pd by IE.

FIG. 5 shows the effect of Ca loading method on the performance of the hybrid catalyst for LPG synthesis from syngas. The reaction conditions were: temperature 290 degrees C., pressure 2.1 MPa, GHSV=1500 h⁻¹, ratio of Cu—Zn—Al/modified Y catalyst=7/9 (by weight—mixed by granular mixing), feed gas included H₂/CO/N₂=64/32/4.

Besides transforming into hydrocarbons, about 46% carbon oxides were converted to CO₂ through the water-gas shift reaction. In addition, trace amounts of methanol and/or DME were also generated. Only CO conversion and LPG selectivity in hydrocarbons are shown in FIG. 5 for clarity of the comparison of the performance of the hybrid catalysts.

The decrease of LPG selectivity over Cu—Zn—Al/IMP-0.5Ca—Pd—Y was similar to that over Cu—Zn—Al/IE-0.5Ca—Pd—Y, and slower than that over Cu—Zn—Al/IE-Pd—Y. On the other hand, the decrease of CO conversion over Cu—Zn—Al/IMP-0.5Ca—Pd—Y was faster than that over Cu—Zn—Al/IE-0.5Ca—Pd—Y and Cu—Zn—Al/IE-Pd—Y. However, they demonstrated much better stability than another hybrid catalyst Cu—Zn—Al/IMP-0.5Ca-IE-Pd—Y. In this arrangement, it was seen that the most preferred way for Ca loading to a Y zeolite was via ion exchange together with Pd.

### NH₃-TPD and TPO-MS

To investigate the relationship of stability, coke deposition and acidity, NH₃-TPD (temperature programmed desorption of NH₃) and TPO-MS (temperature programmed oxidation and mass spectrometric detection) analysis were carried out to characterize the modified Y zeolite before and after reaction. FIG. 6a shows NH₃-TPD profiles of modified Y zeolite before reaction. The two NH₃ desorption peaks at low and high temperature corresponded to the weak and strong acid sites, respectively. Based on the peak area, the strong acid sites decreased to some extent due to the introduction of Ca, especially by the incipient-wetness impregnation method, but the weak acid sites were almost unchanged.

FIG. 6b shows TPO-MS results of modified Y zeolite after reaction. It is believed that coke deposition takes place easily on the strong acid sites. According to NH₃-TPD results in FIG. 6a, it can be understood that the total amount of coke on IE-0.5Ca—Pd—Y and IMP-0.5Ca—Pd—Y was less than that on IE-Pd—Y. For IMP-0.5Ca-IE-Pd—Y, the conversion reaction was only carried out for 24 h. However, coke amount on IMP-0.5Ca-IE-Pd—Y was higher than that on the other three ones which endured 100 h reaction. Without wishing to be bound by any particular theory, it is believed that there was so little Pd exchanged onto Y zeolite after Ca impregnation that the hydrogenation ability of catalyst decreased drastically. The poor hydrogenation ability could not restrain olefins polymerizing to form coke, and then resulted in the quick deactivation of hybrid catalyst Cu—Zn—Al/IMP-0.5Ca-IE-Pd—Y (FIG. 5).

Thus it was considered that the introduction of Ca weakened the strong acid sites of Y zeolite, suppressed coke formation, and thus improved the stability of hybrid catalyst in these examples.

It will be understood that the present invention has been described above purely by way of example, and modification of detail can be made within the scope of the invention.

Each feature disclosed in the description, and (where appropriate) the claims and drawings may be provided independently or in any appropriate combination.

1-40. (canceled)

41. A modified catalyst for use as a dehydration/hydrogenation catalyst in a multi-stage catalyst system for the catalysed production of saturated hydrocarbons from carbon oxides and hydrogen, the modified catalyst comprising:
an acidic substrate comprising an M1-zeolite or M1-silicoalumino phosphate (SAPO) catalyst, where M1 is a metal; and
a modifier including a metal M2,
wherein M2 comprises an alkali metal or alkaline earth metal.

42. A catalyst according to claim 41, wherein the modifier includes a group II metal.

43. A catalyst according to claim 42, wherein the modifier includes Ca.

44. A catalyst according to claim 41 wherein the acidic substrate comprises one or more from the group comprising Y zeolite, β zeolite, ZSM-5, SAPO-5, SAPO-34 and mordenite.

45. A catalyst according to claim 41 wherein M1 comprises a hydrogenation catalyst.

46. A catalyst according to claim 41 wherein M1 comprises one or more from the group comprising Pd Pt, Rh, Ru, and Cu.

47. A multi-stage catalyst system for use as a dehydration/hydrogenation catalyst in the catalysed production of saturated hydrocarbons from carbon oxides and hydrogen, the catalyst system including a first stage comprising a carbon oxide(s) conversion catalyst, and a second stage including a modified catalyst comprising:
an M1-zeolite or M1-SAPO catalyst, where M1 is a metal; and
a modifier including a metal M2,
wherein M2 is an alkali metal or alkaline earth metal.

48. A catalyst system according to claim 47 wherein the carbon oxides conversion catalyst comprises a methanol synthesis catalyst.

49. A hybrid catalyst for the catalysed production of saturated hydrocarbons from carbon oxides and hydrogen, hybrid catalyst including:
a carbon oxide(s) conversion catalyst, and
a modified catalyst according to claim 41.

50. A hybrid catalyst according to claim 49, wherein the carbon oxide(s) conversion catalyst comprises a methanol synthesis catalyst.

51. A hybrid catalyst according to claim 49 wherein the carbon oxide(s) conversion catalyst includes one or more of Cu—ZnO—[Sup], Pd-[Sup] and Zn—Cr-[Sup], where [Sup] is a support composition.

52. A hybrid catalyst according to claim 51, wherein the support composition includes Al₂O₃, SiO₂, and/or zeolite.

53. A hybrid catalyst according to claim 49 wherein the weight percent of modified catalyst in the hybrid catalyst is from about 20% to 80%.

54. A modified catalyst for use in the catalysed production of saturated hydrocarbons from carbon oxides and hydrogen, the modified catalyst comprising:
an M1-SAPO catalyst, where M1 is a metal; and
a modifier including a metal M2,
wherein M2 is an alkali metal or alkaline earth metal.

55. A method of preparing a catalysts according to claim 41.

56. A method according to claim 55, including the step of adding the metal M1 and the modifier substantially simultaneously to the acidic substrate, or adding metal M1 before the modifier.

57. A method of preparing a catalyst for use in the catalysed production of saturated hydrocarbons, the catalyst comprising a metal M1 and an acidic substrate selected from a zeolite and/or a silicoalumino phosphate (SAPO), and a modifier including a metal M2, wherein M2 is an alkali metal or alkaline earth metal, the method including the step of adding the metal M1 and the modifier to the acidic substrate, wherein the metal M1 is added to the acidic substrate before or at substantially the same time as the modifier.

58. A method according to claim 57 wherein the metal M1 and/or the modifier are added to the acidic substrate by an ion exchange method.

59. A method according to claim 58 wherein the temperature of the ion-exchange method is from about 30 to 80 degrees C.

60. A method according to claim 57, wherein the metal M1 and/or the modifier are added to the acidic substrate by an incipient wetness method.

61. A method according to claim 57, wherein after the addition of the metal M1 and the modifier to the acidic substrate, the acidic substrate is heat treated at a temperature between from 450 to 800 degrees C.

62. A method according to claim 57, further including the step of mixing the modified catalyst and a carbon oxide(s) conversion catalyst to form a hybrid catalyst.

63. A method according to claim 62, wherein the carbon oxide(s) conversion catalyst includes a methanol synthesis catalyst.

64. A method according to claim 62 wherein the carbon oxide(s) conversion catalyst includes Cu—ZnO—[Sup], Pd-[Sup] and Zn—Cr-[Sup], where [Sup] is a support composition.

65. A method according to claim 64, wherein the support composition includes Al₂O₃, SiO₂, and/or zeolite.

66. A method according to claim 62, wherein the weight percent of modified catalyst in the hybrid catalyst is from about 20% to 80%.

67. A process for the catalysed production of saturated hydrocarbons using a catalyst system including a modified dehydration/hydrogenation catalyst, wherein the modified catalyst comprises:
an M1-zeolite or M1-SAPO catalyst, where M1 is a metal, and
a modifier including a metal M2, wherein M2 is an alkali metal or alkaline earth metal.

68. A process according to claim 67, wherein the catalyst system further comprises a carbon oxide(s) conversion catalyst.

69. A process according to claim 67 wherein the catalyst system includes a hybrid catalyst as defined above.

70. A process according to claim 67 having a reaction temperature between from about 260 to 400 degrees C.

71. A process according to claim 67 having a reaction pressure between from about 0.5 to 6.0 MPa.

72. A process according to claim 67, having a gas space velocity from about 500 to 6000 h⁻¹.

73. A process according to claim 67 wherein the carbon oxide(s) conversion catalyst is in a first reaction stage separate from a second reaction stage including the modified catalyst.

74. A process according to claim 67 further including the step of carrying out a regeneration treatment of the modified catalyst.

75. A process according to claim 74, wherein the regeneration treatment includes heating the catalyst to a temperature of at least 500 degrees C.

76. A process according to claim 74, further including the step of separating at least part of the modified catalyst from a catalyst composition before carrying out the regeneration treatment.

77. A process according to claim 67 wherein the product includes C₃ and/or higher saturated hydrocarbons.

78. A process according to claim 67 including a catalyst as defined above and/or wherein the catalyst is obtained as defined above.

79. A catalyst obtained or obtainable by a method according to claim 55.
